(19) United States
(12) Patent Application Publication
(10) Pub. No.: US 2015/0313501 A1
(43) Pub. Date: Nov. 5, 2015

(54) HIGH RESOLUTION ELECTRO-ANATOMIC MAPPING USING MULTIPLE BIOPOTENTIAL SENSORS AND ASSOCIATED SIGNAL PROCESSING AND DIGITIZATION IN THE CATHETER TIP

(71) Applicant: Magnetecs Corporation, Inglewood, CA (US)

(72) Inventor: Josh Shachar, Santa Monica, CA (US)

(73) Assignee: MAGNETECS CORPORATION, Inglewood, CA (US)

(21) Appl. No.: 14/797,020

(22) Filed: Jul. 10, 2015

Related U.S. Application Data
(63) Continuation-in-part of application No. 13/549,341, filed on Jul. 13, 2012.
Application No. 13/549,341 is a continuation-in-part of application No. 13/621,727, filed on Sep. 17, 2012.

(51) Int. Cl. A61B 5/0428; A61B 5/04; A61B 5/00; A61B 5/042

(52) U.S. Cl. CPC A61B 5/04284; A61B 5/0422; A61B 5/04017; A61B 5/6852

(57) ABSTRACT

An apparatus for sensing an electrophysiological biopotential signal in combination with an external control circuit includes a catheter having a tip portion, an analog front-end sensor array in the tip portion of the catheter communicated with at least a first electrode in the tip portion of the catheter, and an analog signal processing integrated circuit in the tip portion of the catheter communicated with analog front-end sensor array.

### RELATED CASES

The present application is a continuation in part application of application Ser. No. 13/549,341 filed on Jul. 13, 2012, entitled METHOD AND APPARATUS FOR MAGNETICALLY GUIDED CATHETER FOR RENAL DENERVATION EMPLOYING MOSFET SENSOR ARRAY, and application Ser. No. 13/621,727, filed on Sep. 17, 2012, entitled METHOD AND APPARATUS FOR MEASURING BIOPOTENTIAL AND MAPPING EPHAPTIC COUPLING EMPLOYING A CATHETER WITH MOSFET SENSOR ARRAY under 35 USC 120 and incorporated herein by reference.

### BACKGROUND

1. Field of the Technology

The invention relates to the field of apparatus and methods of sensing biopotential using catheters.

2. Description of the Relevant Art

An electro-anatomic map is a data set of biopotential measurements taken over time at various locations within or near a body region. In the research environment, the goal is to facilitate the creation and validation of ever more accurate models of the electrophysiology of disorders for the sake of improved clinical diagnostics. In clinical applications, the goal is to supplement the overall evidence so as to alto the medical team to formulate the correct diagnosis, and often also to facilitate the treatment.

The quality and usefulness of the map depends on the sensor type, sensor array pitch and size, sampling rate and synchronization, and measurement accuracy, all relative to the characteristics of the organ or tissue being examined. For example, cardiac arrhythmia is characterized as occurring over a relatively large volume in a dynamic, complex temporospatial process wherein the electrical activity at a given location is the sum of near-field myocardial activation, ephaptic coupling, magnetic heart vector effects, far-field neuromuscular activity, and emissions from operating room and surgical equipment. Some of these contributions to each discrete measurement are part of the “signal” of interest, the remainder are “noise” or “artifacts,” Because of the dynamics, a useful map cannot be constructed by moving a single sensor about, but rather requires the synchronous or quasi-synchronous sampling of a closely spaced array of sensors. These considerations universally apply to many metrology and signal processing applications, including any approach to electroanatomic mapping.

### BRIEF SUMMARY

The illustrated embodiments of the invention include an apparatus for sensing, an electrophysiological biopotential signal in combination with an external control circuit including a catheter having a tip portion, an analog, front end sensor array in the tip portion of the catheter communicated with at least a first electrode in the tip portion of the catheter, and an analog signal processing integrated circuit in the tip portion of the catheter communicated with analog front-end sensor array.

In another embodiment the apparatus further includes at least a second electrode in the tip portion of the catheter corresponding to the at least first electrode to comprise an electrode pair, the electrode pair being communicated with the analog processing integrated circuitry.

The apparatus further includes a MOSFET circuit in the tip portion of the catheter communicated with the at least first electrode, the MOSFET circuit being communicated with the analog signal processing integrated circuit.

In one embodiment the MOSFET circuit in the tip portion of the catheter communicated with the electrode pair, the MOSFET circuit is communicated with the analog signal processing integrated circuit.

The analog signal processing integrated circuit includes analog circuitry and a digital signal processor in the tip portion of the catheter communicated with the analog circuitry to control the analog circuitry according to external commands and/or locally and adaptively based signal properties within the catheter.

The external control circuit communicates with the digital signal processor in the tip portion of the catheter to provide digital processing of the electrophysiological biopotential signal.

The analog circuitry comprises a low noise differential amplifier having an input coupled to the at least first electrode, a high pass filter having an input coupled to an output of the differential amplifier, a programmable amplifier having an input coupled to an output of the high pass filter, a low pass filter having an input coupled to an output of the programmable amplifier, an analog-to-digital converter having an input coupled to an output of the low pass filter, and the analog-to-digital converter having an output coupled to an input of the digital signal processor.

The digital signal processor in the tip portion of the catheter is communicated with the analog circuitry to control the analog circuitry, where the low-noise amplifier has a variable gain/attenuation and where the digital signal processor controls the gain/attenuation of the low-noise amplifier to keep the signal linear and within the dynamic range of the rest of the processing chain.

The digital signal processor in the tip portion of the catheter is communicated with the analog circuitry to control the analog circuitry, where the high pass filter has a variable corner frequency and where the digital signal processor controls the corner frequency of high-pass filter within predetermined frequency range with a predetermined maximum stop-band attenuation.

The digital signal processor in the tip portion of the catheter is communicated with the analog circuitry to control the analog circuitry, where the programmable amplifier has a variable gain and where the digital signal processor controls the gain of programmable amplifier within a predetermined range in predetermined steps.

The digital signal processor in the tip portion of the catheter is communicated with the analog circuitry to control the analog circuitry, where the low pass filter has a variable corner frequency and where the digital signal processor controls the corner frequency of the low-pass filter within a predetermined range with a predetermined stop-band attenuation.

The digital signal processor in the tip portion of the catheter is communicated with the analog circuitry to control the analog circuitry, where the analog-to-digital converter has a variable sampling rate and where the digital signal processor controls the sampling rate of analog-to-digital converter with a predetermined number of effective noise-free bits up to a predetermined sampling frequency.

The illustrated embodiments also include an apparatus for sensing an electrophysiological biopotential signal including a catheter having a tip portion, a plurality of electrodes in the tip portion of the catheter, a corresponding plurality of analog front-end sensor circuits in the tip portion of the catheter each communicated with at least one of the plurality of electrodes, and a corresponding plurality of analog signal processing integrated circuits each communicated with a corresponding one of the plurality of analog front-end sensor circuits.

The corresponding plurality of analog front-end sensor circuits in the tip portion of the catheter are each communicated with only one of the plurality of electrodes.

The plurality of electrodes are configured into a plurality of pairs of electrodes in the tip portion of the catheter and where the corresponding plurality of analog front-end sensor circuits in the tip portion of the catheter are each communicated with one pair of the plurality of pairs of electrodes.

The corresponding plurality of analog front-end sensor circuits each comprise a MOSFET sensing circuit in the tip portion of the catheter, the corresponding plurality of analog signal processing integrated circuits in the tip portion of the catheter each communicated with at least one of the plurality of MOSFET sensing circuits.

The illustrated embodiments of the invention also extend to a method for sensing an electrophysiological biopotential signal the steps of coupling the electrophysiological biopotential signal to at least a first electrode in a tip portion of the catheter, sensing the coupled electrophysiological biopotential signal with an analog front-end sensor circuit in the tip portion of the catheter communicated with the at least first electrode, and processing the sensed analog electrophysiological biopotential signal into a digital signal with an analog signal processing integrated circuit in the tip portion of the catheter communicated.

The step of sensing the electrophysiological biopotential signal with at least a first electrode in a tip portion of the catheter includes sensing the electrophysiological biopotential signal with an electrode pair in a tip portion of the catheter.

The method further includes the step of sensing the electrophysiological biopotential signal with at least a first electrode in a tip portion of the catheter by using a MOSFET circuit in the tip portion of the catheter communicated with the at least first electrode, the MOSFET circuit being communicated with the analog processing integrated circuit.

The method further includes the step of controlling the analog signal processing integrated circuit using a digital signal processor therein according to external commands and/or locally and adaptively based signal properties within the catheter.

The method further includes the step of digitally processing the electrophysiological biopotential signal using an external control circuit communicated with the digital signal processor in the tip portion of the catheter.

The step of controlling the analog signal processing integrated circuit using a digital signal processor therein includes controlling gain/attenuation of a low-noise amplifier in the analog signal processing integrated circuit to keep the signal linear and within the dynamic range of the rest of the processing chain, controlling a corner frequency of a high-pass filter in the analog signal processing integrated circuit within predetermined frequency range with a predetermined maximum stop-band attenuation, controlling gain of a programmable amplifier in the analog signal processing integrated circuit within a predetermined range in predetermined steps, controlling a corner frequency of a low-pass filter in the analog signal processing integrated circuit within a predetermined range with a predetermined stop-band attenuation, and controlling a sampling rate of an analog-to-digital converter in the analog signal processing integrated circuit with a predetermined number of effective noise-free bits up to a predetermined sampling frequency.

The illustrated embodiments also include an apparatus for sensing an electrophysiological biopotential signal including a catheter having a tip portion, a plurality of electrodes in the tip portion of the catheter, a corresponding plurality of MOSFET sensing circuits in the tip portion of the catheter coupled to the plurality of electrodes in the tip portion of the catheter, and a corresponding plurality of analog signal processing integrated circuits in the tip portion of the catheter each communicated with at least one of the plurality of MOSFET sensing circuits.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a block diagram of a prior art analog circuit for processing an ECG signal.

FIG. 2 is a block diagram a multiple-channel differential 5 electrode pair test-bed “local amplifier” or 32 pair electrode production catheter using an array of multiple miniature dual instrumentation amplifier ICs in the tip portion of the catheter, with the two inputs of each IC connected to corresponding adjacent electrode pairs.

FIG. 3 is a schematic diagram of a MOSFET detector circuit used in the catheter for direct connection to an electrode pair. FIG. 3 further depicts a combination of MOSFET sensor in differential mode architecture.

FIG. 4 is a block diagram of a dual biochip (bipolar configuration) of the illustrated embodiment of the analog front-end and its digital processing section integrated on a common substrate and placed in the tip portion of a production catheter and shown coupled to an electrode pair or MOSFET circuit of FIG. 3.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

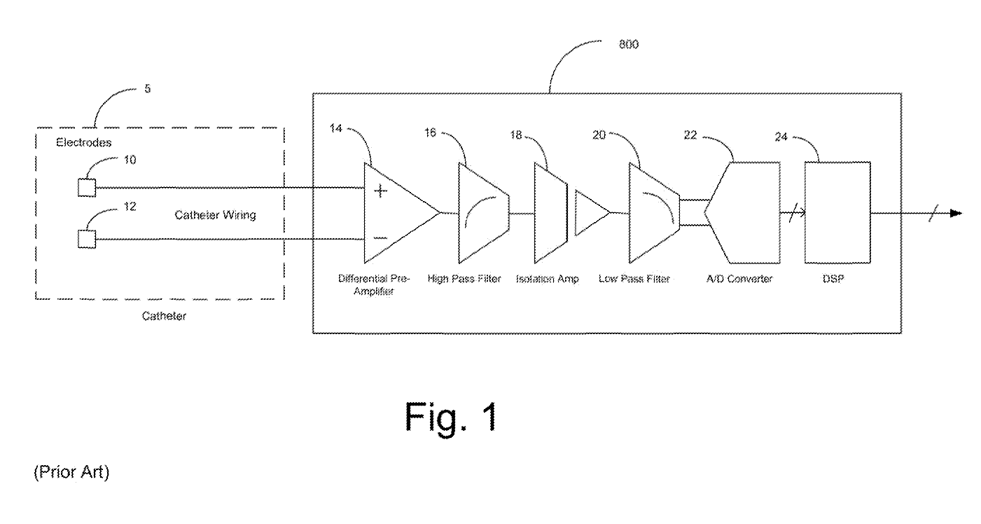

FIG. 1 is a block diagram of a conventional intracardial ECG signal path from a pair of ECG electrodes 10, 12 disposed on or in a cardiac catheter and provided to the input of a differential amplifier 14. The output is processed through high-pass filter 16 through an isolation amplifier 18 to a low pass filter 20 and thence to an analog-to-digital converter via power and control 72. The digitized prepared signal is then coupled for analysis and display to a computer or digital processor 24. For any measurement path consisting of a series of processing stages and interconnects, such as that shown in Error! Reference source not found, each path element in the analog domain, such as from electrodes 10, 12 to the input of the A-to-D converter 22, has a noise factor F=SNRᵢₙ/SNRₒᵤₜ, where SNRᵢₙ is the signal-to-noise ratio at the element input and SNRₒᵤₜ is the signal-to-noise ratio at the element output. Lower values of F indicate better performance. The noise factor of the overall signal path is dominated by the noise factor of the first element, such as the electrodes 10, 12, to the extent that the noise at the output of this element is within the passband of the path. In the case of the conventional ECG signal path shown in Erorr! Reference source not found, the electrical signal level at the catheter electrodes may be as low as 1 μV, while considerable environmental electrical noise is coupled to the long wire run through the catheter, even if the wiring is twisted and shielded. The external or following circuitry cannot overcome the poor SNR present at its input.

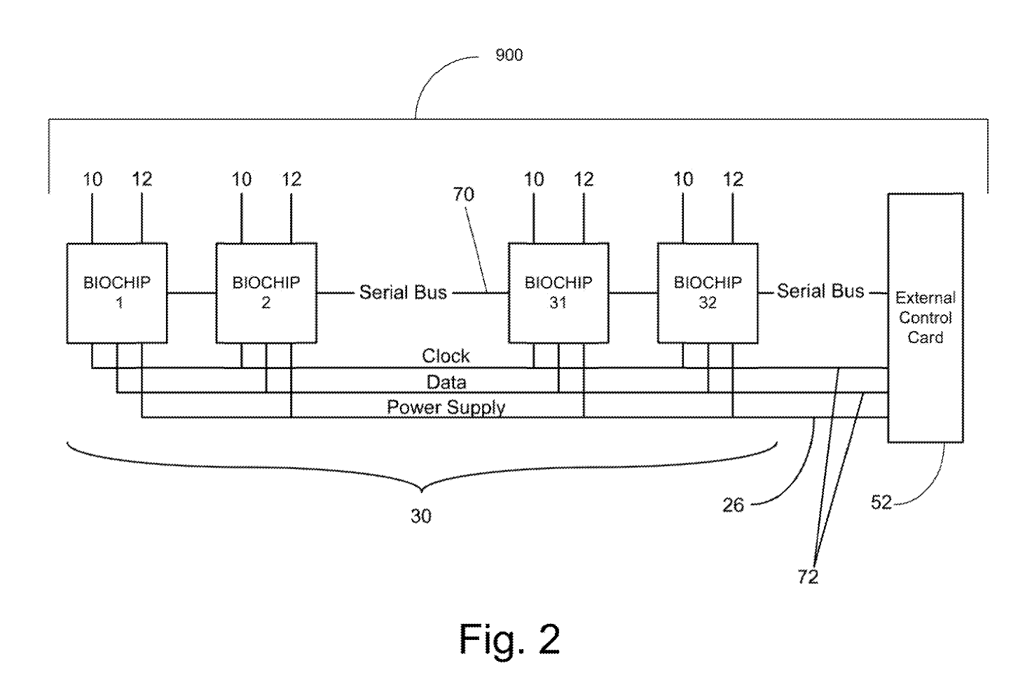

To demonstrate the degrading effect of passive electrodes and catheter wiring on signal quality, what is disclosed below is a multiple-channel differential (decapolar −5 electrode pairs or a 32 electrode pair production model) test-bed “local amplifier” catheter 26 in FIG. 2 using an array of multiple miniature dual instrumentation amplifier ICs (Analog Devices AD8235) 28(1)-28(32) in the tip portion 30 of catheter 26 with the two inputs of each IC connected to corresponding adjacent electrode pairs 10(1)-10(32), 12(1)-12(32). A 100 Hz sine wave signal attenuated to less than 10 μVₚₑₐₖ₋ₜₒ₋ₚₑₐₖ was applied differentially to one channel, and also to an electrode pair of an identically structured passive decapolar catheter (not shown) having the same tip-to-output wiring configuration, but without the dual instrumentation amplifier ICs. For the passive catheter, the output was amplified by an external amplifier having a voltage gain of 128. The signal-to-noise (SNR) was −50 dB. For the active catheter 26, the local, in-tip amplifier voltage gain was set to 100. The SNR of the catheter output was −13 dB. This demonstrates the great improvement obtained by moving the gain block to the catheter tip portion 30.

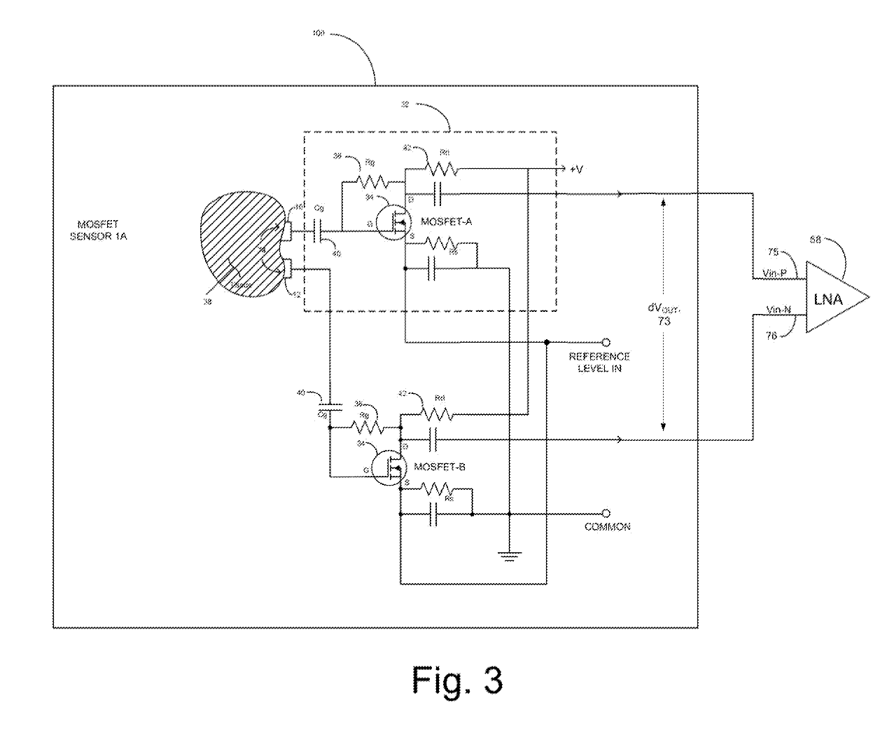

One way to achieve voltage gain at the catheter tip is to use a MOSFET sensor as described in U.S. Pat. No. 7,869,854, incorporated herein by reference, assigned to Magnetecs Corp. and illustrated in FIG. 3. The sensor 32 includes an N-channel MOSFET 34 (metal-oxide-silicon field-effect transistor) that is operated in drain feedback bias mode. MOSFET drain current is a relatively linear function of gate-source voltage. Since gate current is negligible, Vgs=Vds even though resistor 36, R_{g}, has a large (1 megohm) resistance. In this bias mode, at turn on, the gate voltage is pulled up until the drain current pulls drain voltage down to a stable quiescent operating point.

Gate voltage then tracks changes in biopotential, dVᵢₙₚᵤₜ, 74, of tissue 38 via capacitive coupling thru capacitor 40, Cg, thereby modulating drain current to provide the voltage gain seen across drain resistor 42, Rd. The following variations are feasible within a bipolar (differential) input configuration, as shown by reference designator 73, dV_{Output}, as shown in FIG. 3. MOSFET 34 responds to the differential biopotential dVᵢₙₚᵤₜ 74, between the two electrodes 10, 12. A DC offset is allowed to accumulate across capacitive coupling 44, Cs, or an externally controlled reference level may be used to reduce or eliminate this offset. In the unipolar (single-ended) input configuration, capacitor 44, Cs, and its associated electrode 12 are eliminated. A DC offset is allowed to accumulate across capacitive coupling 40, Cg, or an externally controlled reference level may be used to reduce or eliminate this offset.

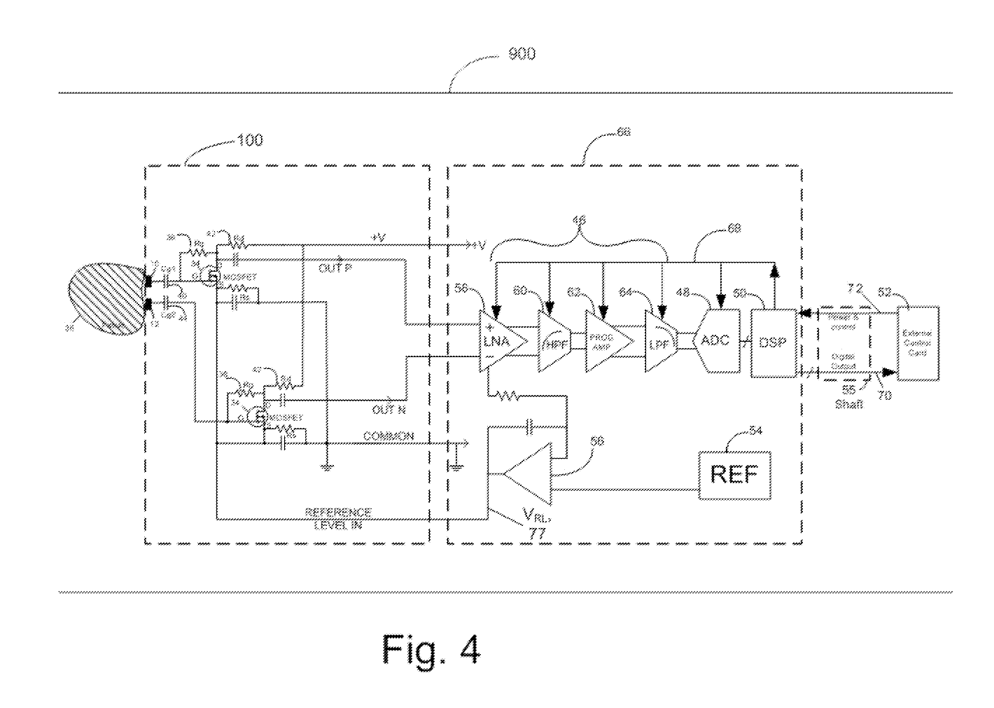

FIG. 4 is a diagram of the overall system 900 including 1) analog front-end sensor array 100, 2) BioChip 66 and 3) external control card 52. A plurality of analog front-end sensor arrays 100 and corresponding BioChips 66 are included and disposed in the distal portion of the sensing catheter 5, which has been modified according to the teachings of the illustrated embodiments of the invention. A further improvement in signal fidelity may be achieved by incorporating the entire analog signal processing path or circuit 46, the A/D converter 48, and a programmable DSP (digital signal processing) core 50 in an integrated circuit (IC) 66 as shown in FIG. 4. The analog signal processing path or circuit 46 takes its input from an analog front-end sensor array 100, which is coupled to a BioChip 66.

The analog signal processing path 46 includes a low noise differential amplifier 58 having its input coupled to the electrodes 10, 12 and/or MOSFET sensing circuit 32. The amplifier 58 is coupled to the input of a high pass filter 60 followed by a programmable amplifier 62, a low pass filter 64 then coupled to A/D converter 48. A reference voltage 54 is provided to both A/D converter 48 and to reference level generator (virtual ground) 56 having its output coupled to MOSFET sensing circuit 32. Each of these elements are included in a custom integrated circuit 66 is called a Bio Chip 66. It is fabricated as a wafer-level chip-scale package using the SMIC CMOS 55 nm, low-power silicon process with the physical dimensions are 2.2×2.9×0.8 mm for inclusion on or in tip portion 30 of the catheter 26 illustrated in FIG. 2.

The on-board, externally-programmable DSP (digital signal processing) core 50 is capable of performing a 2048-tap linear filter (finite impulse response), and up to 4096-bin Fast Fourier Transform. Core 50 controls the analog chain by communication with each of the circuit elements in the chain through a control bus 68. The gain/attenuation of Low-noise amplifier 58 is adjusted to keep the signal linear and within the dynamic range of the rest of the processing chain. The corner frequency of high-pass filter 60 is adjusted within 0 to 0.5 Hz range, with 80 dB maximum stop-band attenuation. The gain of programmable amplifier 62 is adjusted from 18 to 76 dB in 0.63 dB steps. The corner frequency of low-pass filter 64 set to 32, 64, 128, or 256 Hz, with 80 dB stop-band attenuation. The operation of analog-to-digital converter 48 is programmable with 14 effective noise-free bits up to 16 kHz.

The Bio Chip inputs, Vin-N, 76, Vin-P, 75, and V_{RL}, 77, can be wired directly to catheter electrodes either differentially from two electrodes or unipolar from one electrode with Vin-N connected to V_{RL}). The Bio Chip inputs can also be connected to a MOSFET sensor circuit 32 as shown in FIG. 3 with the same differential or single-ended options available. The reference level generator 56 in the Bio Chip 66 is an integrator that adjusts the sensor reference voltage to keep its common mode output voltage, Vcm, close to Bio Chip internal reference voltage generator 54.

Each catheter can include up to 32 separate Bio Chips 66 that communicate with the external control card or circuit 52 via a daisy-chained synchronous serial communication bus 70 similar to the de facto industry standard SPI bus as shown in FIG. 2. The chips 66 along each catheter spline of a basket catheter used in cardiac ablation, for example, are concatenated to each other, with the splines in a multi-spine catheter concatenated end-to-end. The bus clock operates in the range of 0.2 MHz to 0.4 MHz and is provided by external control card 52 on signal traces 72 along with a data bus from Bio Chips 66 to the external control card 52.

Digitizing the individual electrograms in the catheter tip portion 30 and delivering the data digitally through the catheter 26 completely eliminates noise corruption between the catheter tip portion 30 and the external processing circuit 52 and allows this circuit 52 to be located away from the operating table without loss of signal fidelity. Overall digital signal processing between the ADC outputs and the data presentation to the medical team is divided between the in-catheter DSP 50 and external DSP engines included in control card 52. Each in-catheter DSP 50 performs the functions needed to configure the associated in-tip analog stages according to external co wands and/or locally and adaptively based signal properties of the channel or catheter. The external DSP 52 operates on the output of each channel to generate ensemble averages as required a id synthesizes the outputs of all of catheter channels to extract, analyze, and display the temporospatial electro-anatomic data and map needed by the operating team.

Many alterations and modifications may be made by those having ordinary skill in the art without departing from the spirit and scope of the embodiments, Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in, the context of this specification as including more than cine meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the embodiments.

We claim:

1. An apparatus for sensing an electrophysiological biopotential signal in combination with an external control circuit comprising:
a catheter having a tip portion;
an analog front-end sensor array in the tip portion of the catheter communicated with at least a first electrode in the tip portion of the catheter; and
an analog signal processing integrated circuit in the tip portion of the catheter communicated with analog front-end sensor array.

2. The apparatus of claim 1 further comprising at least a second electrode in the tip portion of the catheter corresponding to the at least first electrode to comprise an electrode pair, the electrode pair being communicated with the analog processing integrated circuitry.

3. The apparatus of claim 1 further comprising a MOSFET circuit in the tip portion of the catheter communicated with the at least first electrode, the MOSFET circuit being communicated with the analog signal processing integrated circuit.

4. The apparatus of claim 2 further comprising a MOSFET circuit in the tip portion of the catheter communicated with the electrode pair, the MOSFET circuit being communicated with the analog signal processing integrated circuit.

5. The apparatus of claim 1 where the analog signal processing integrated circuit comprises analog circuitry and a digital signal processor in the tip portion of the catheter communicated with the analog circuitry to control the analog circuitry according to external commands and/or locally and adaptively used signal properties within the catheter.

6. The apparatus of claim 5 where the external control circuit communicates with the digital signal processor in the tip portion of the catheter to provide digital processing of the electrophysiological biopotential signal.

7. The apparatus of claim 5 where the analog circuitry comprises a low noise differential amplifier having an input coupled to the at least first electrode, a high pass filter having an input coupled to an output of the differential amplifier, a programmable amplifier having an input coupled to an output of the high pass filter, a low pass filter having an input coupled to an output of the programmable amplifier, an analog-to-digital converter having an input coupled to an output of the low pass filter, and the analog-to-digital converter having an output coupled to an input of the digital signal processor.

8. The apparatus of claim 7 where the digital signal processor in the tip portion of the catheter is communicated with the analog circuitry to control the analog circuitry, where the low-noise amplifier has a variable gain/attenuation and where the digital signal processor controls the gain/attenuation of the low-noise amplifier to keep the signal linear and within the dynamic range of the rest of the processing chain.

9. The apparatus of claim 7 where the digital signal processor in the tip portion of the catheter is communicated with the analog circuitry to control the analog circuitry, where the high pass filter has a variable corner frequency and where the digital signal processor controls the corner frequency of high-pass filter within predetermined frequency range with a predetermined maximum stop-band attenuation.

10. The apparatus of claim 7 where the digital signal processor in the tip portion of the catheter is communicated with the analog circuitry to control the analog circuitry, where the programmable amplifier has a variable gain and where the digital signal processor controls the gain of programmable amplifier within a predetermined range in predetermined steps.

11. The apparatus of claim 7 where the digital signal processor in the tip portion of the catheter is communicated with the analog circuitry to control the analog circuitry, where the low pass filter has a variable corner frequency and where the digital signal processor controls the corner frequency of the low-pass filter within a predetermined range with a predetermined stop-band attenuation.

12. The apparatus of claim 7 where the digital signal processor in the tip portion of the catheter is communicated with the analog circuitry to control the analog circuitry, where the analog-to-digital converter has a variable sampling rate and where the digital signal processor controls the sampling rate of analog-to-digital converter with a predetermined number of effective noise-free bits up to a predetermined sampling frequency.

13. An apparatus for sensing an electrophysiological biopotential signal comprising:
a catheter having a tip portion;
a plurality of electrodes in the tip portion of the catheter;
a corresponding plurality of analog front-end sensor circuits in the tip portion of the catheter each communicated with at least one of the plurality of electrodes; and
a corresponding plurality of analog signal processing integrated circuits each communicated with a corresponding one of the plurality of analog front-end sensor circuits.

14. The apparatus of claim 13 where the corresponding plurality of analog front-end sensor circuits in the tip portion of the catheter are each communicated with only one of the plurality of electrodes.

15. The apparatus of claim 13 where the plurality of electrodes are configured into a plurality of pairs of electrodes in the tip portion of the catheter and where the corresponding plurality of analog front-end sensor circuits in the tip portion of the catheter are each communicated with one pair of the plurality of pairs of electrodes.

16. The apparatus of claim 13 where the corresponding plurality of analog front-end sensor circuits each comprise a MOSFET sensing circuit in the tip portion of the catheter, the corresponding plurality of analog signal processing integrated circuits in the tip portion of the catheter each communicated with at least one of the plurality of MOSFET sensing circuits.

17. A method for sensing an electrophysiological biopotential signal comprising:
coupling the electrophysiological biopotential signal to at least a first electrode in a tip portion of the catheter;
sensing the coupled electrophysiological biopotential signal with an analog front-end sensor circuit in the tip portion of the catheter communicated with the at least first electrode; and
processing the sensed analog electrophysiological biopotential signal into a digital signal with an analog signal processing integrated circuit in the tip portion of the catheter communicated.

18. The method of claim 17 where sensing the electrophysiological biopotential signal with at least a first electrode in a tip portion of the catheter comprises sensing the electrophysiological biopotential signal with an electrode pair in a tip portion of the catheter.

19. The method of claim 17 further comprising sensing the electrophysiological biopotential signal with at least a first electrode in a tip portion of the catheter by using a MOSFET circuit in the tip portion of the catheter communicated with the at least first electrode, the MOSFET circuit being communicated with the analog processing integrated circuit.

20. The method of claim 17 further comprising controlling the analog signal processing integrated circuit using a digital signal processor therein according to external commands and/or locally and adaptively based signal properties within the catheter.

21. The method of claim 20 further comprising digitally processing the electrophysiological biopotential signal using an external control circuit communicated with the digital signal processor in the tip portion of the catheter.

22. The method of claim 20 where controlling the analog signal processing integrated circuit using a digital signal processor therein comprises controlling gain/attenuation of a low-noise amplifier in the analog signal processing integrated circuit to keep the signal linear and within the dynamic range of the rest of the processing chain, controlling a corner frequency of a high-pass filter in the analog signal processing integrated circuit within predetermined frequency range with a predetermined maximum stop-band attenuation, controlling gain of a programmable amplifier in the analog signal processing integrated circuit within a predetermined range in predetermined steps, controlling a corner frequency of a low-pass filter in the analog signal processing integrated circuit within a predetermined range with a predetermined stop-band attenuation, and controlling a sampling rate of an analog-to-digital converter in the analog signal processing integrated circuit with a predetermined number of effective noise-free bits up to a predetermined sampling frequency.

23. An apparatus for sensing an electrophysiological biopotential signal comprising:
a catheter having a tip portion;
a plurality of electrodes in the tip portion of the catheter;
a corresponding plurality of MOSFET sensing circuits in the tip portion of the catheter coupled to the plurality of electrodes in the tip portion of the catheter; and a corresponding plurality of analog signal processing integrated circuits in the tip portion of the catheter each communicated with at least one of the plurality of MOSFET sensing circuits.
